(19) United States
(12) Patent Application Publication
(10) Pub. No.: US 2008/0287833 A1
(43) Pub. Date: Nov. 20, 2008

(54) Method of evaluating gastroparesis using an ingestible capsule

(76) Inventors: John R. Semler, Williamsville, NY (US); Braden Kuo, Newton, MA (US)

Correspondence Address: PHILLIPS LYTLE LLP;INTELLECTUAL PROPERTY GROUP, 3400 HSBC CENTER, BUFFALO, NY, 14203-3509, US

(21) Appl. No.: 11/899,540

(22) Filed: Sep. 6, 2007

Related U.S. Application Data
(60) Provisional application No. 60/930,451, filed on May 16, 2007.

(51) Int. Cl. A61B 5/00

(52) U.S. Cl. 600/593; 128/898

(57) ABSTRACT

A method of evaluating gastroparesis comprising the steps of providing an ingestible capsule having a pH sensor and a pressure sensor, having a subject ingest the capsule, recording pH measurements from the pH sensor as a function of time as the capsule moves through at least a portion of the gastrointestinal tract of the subject, recording pressure measurements from the pressure sensor as a function of time as the capsule moves through at least a portion of the gastrointestinal tract of the subject, determining the capsule's location at a first position in the gastrointestinal tract, deriving a pressure pattern as a function of time and the pressure measurements, providing a reference pressure pattern, and analyzing the subject's pressure pattern relative to the reference pressure pattern to evaluate the subject with respect to gastroparesis. The location may be the junction between the stomach and the small bowel of the subject. The pressure pattern may be the number of contractions relative to a baseline over a given time interval or area under the curve of pressure measurements over a given time interval.

### CROSS-REFERENCE TO RELATED APPLICATIONS

This application claims the benefit of U.S. Provisional Patent Application No. 60/930,451, filed May 16, 2007. The entire content of such application is incorporated by reference herein.

### TECHNICAL FIELD

The present invention relates generally to ingestible capsules and, more particularly, to a process for evaluating a subject for gastroparesis with an ingested capsule passing through the digestive tract of the subject.

### BACKGROUND ART

Ingestible capsules are well-known in the prior art. Such capsules are generally small pill-like devices that can be ingested or swallowed by a patient. It is known that such capsules may include one or more sensors for determining physiological parameters of the gastrointestinal tract, such as sensors for detecting temperature, pH, pressure and the like.

It is also known that certain physiological parameters may be associated with regions of the gastrointestinal tract. For example, a 1988 article entitled “Measurement of Gastrointestinal pH Profiles in Normal Ambulant Human Subjects” discloses pH measurements recorded by a capsule passing through the gastrointestinal tract. It is know that pH has been correlated with transitions from the stomach to the small bowel (gastric emptying).

Gastroparesis, also known as delayed gastric emptying, is a condition characterized by multiple symptoms, including nausea, vomiting, bloating, abdominal pain or discomfort and early satiety. Diagnosing gastroparesis is traditionally determined from a combination of symptom assessment and gastric emptying scintigraphy. Gastro duodenal manometry may also be performed to provide further evidence of the condition. Gastro duodenal manometry is an invasive, catheter-based system in which a manometry probe is inserted through a patient's nose or mouth into the GI Tract. The manometry probe usually has a suite of pressure sensors located at fixed positions along its length. These pressure sensors detect and send contraction amplitude and frequency data through connected wires to an external recording device. For placement of the probe, this technique is uncomfortable for the patient and requires the patient to be sedated and physically connected to the detector. Besides being highly uncomfortable, the manometry measurement system directly impacts the normal functioning of the patient, which may skew the manometry results.

An additional method of diagnosing gastroparesis is the use of gastric scintigraphy. This method requires a patient to ingest a meal which contains a known amount of a radioactive compound. Isotope imaging is then used to determine the amount of radioactive matter remaining in the stomach. Physicians take images at times consistent with local standards. In general, if at two hours more than 50% of the radioactive tracer is present, or more than 10% is present after 4 hours, the patient is diagnosed as gastroparetic. This method has numerous drawbacks, including requiring the use of radioactive material, requiring the patient to remain at the test site for at least four hours, a lack of standardization, and requiring patients to stop using certain medications resulting in changes in the patient's normal daily functioning.

Thus, there is need for a less invasive method for diagnosing gastroparesis.

### DISCLOSURE OF THE INVENTION

With parenthetical reference to corresponding parts, portions or surfaces of the disclosed embodiment, merely for the purposes of illustration and not by way of limitation, the present invention provides an improved method for diagnosing gastroparesis comprising the steps of providing an ingestible capsule having a pH sensor and a pressure sensor, having a subject ingest the capsule, recording pH measurements from the pH sensor as a function of time as the capsule moves through at least a portion of the gastrointestinal tract of the subject, recording pressure measurements from the pressure sensor as a function of time as the capsule moves through at least a portion of the gastrointestinal tract of the subject, determining the capsules location at a position in the gastrointestinal tract, deriving a pressure pattern as a function of time and the pressure measurements, providing a reference pressure pattern, and analyzing the subject's pressure pattern relative to the reference pressure pattern to evaluate the subject with respect to gastroparesis.

The location may be the junction between the stomach and the small bowel of the subject. The pressure pattern may be the number of contractions relative to a baseline over a given time interval, the area under the curve of pressure measurements for a given time interval, or amplitude. The step of evaluating the subject may comprise diagnosing gastroparesis. The step of analyzing the pressure pattern may comprise the step of determining if the subject's pressure pattern is significantly diminished from or lower than the reference pressure pattern, wherein the reference pressure pattern is derived from a healthy control group. The subject's pressure pattern may be at least ten percent less than the reference pressure pattern. The subject time may be a period of time that the capsule resides in the stomach of the subject or a period of time extending from about one hour before the capsule is determined to be at the position to the time the capsule is determined to be at the position. The step of analyzing the subject's pressure pattern relative to the reference pressure pattern to evaluate the subject with respect to gastroparesis may comprise the step of comparing the subject's pressure pattern to the reference pressure pattern for three consecutive twenty minute intervals before and after the capsule is determined to be at the position.

The method may further comprise the steps of determining transit time between a first location and a second location, and evaluating the transit time relative to a reference transit time. The first location may be the point at which the capsule is ingested by the subject and the second location may be the junction between the stomach and the small bowel of the subject. The step of evaluating the transit time relative to a reference transit time may comprise the step of determining whether the transit time is greater than or less than the reference transit time, and the reference transit time may be about five hours.

The step of deriving a pressure pattern as a function of time and the pressure measurements may comprise the step of conditioning the recorded pressure measurements. The conditioning may comprise the step of normalizing the pressure measurements by applying a baseline compensation, and the baseline may be about 3 mmHg. The conditioning may comprise the steps of filtering out data points in the pressure measurements above an upper limit and filtering out data points in the pressure measurements below a lower limit, and the upper limit may about 200 mmHg and the lower limit may be about 9 mmHg.

The method may further comprise the steps of deriving a pH pattern as a function of time and the pH measurements and analyzing the pH pattern for the subject and the pressure pattern for the subject relative to a pH reference pattern and a pressure reference pattern to determine the capsule's location at a second position. The method may further comprise the steps of determining transit time between the first position and the second position, and evaluating the transit time relative to a reference transit time. The first position may be a junction between the stomach and the small bowel of the gastrointestinal tract of the subject and the second location may be a junction between the ileum and the caecum of the gastrointestinal tract of the subject. The first position may be the junction between the ileum and the caecum of the gastrointestinal tract of the subject and the second location may be the point at which the capsule is discharged from the gastrointestinal tract of the subject.

The method may further comprise the steps of deriving a second pressure pattern different from the first pressure pattern as a function of time and the pressure measurements, providing a second reference pressure pattern, and analyzing the second pressure pattern variations for the subject relative to the second reference in determining the capsule's location at the first position. The first pressure pattern may be frequency of contractions relative to a baseline over a given time interval and the second pressure pattern may be motility index. The method may further comprise the steps of deriving a second pressure pattern different from the first pressure pattern as a function of time and the pressure measurements, providing a second reference pressure pattern, and analyzing the second pressure pattern variations for the subject relative to the second reference in determining the capsule's location at the second position. The first pressure pattern may be frequency of contractions relative to a baseline over a given time interval and the second pressure pattern may be motility index. The step of determining the capsules location at a first location in the gastrointestinal tract may comprise the steps of providing a reference pH or a reference degree of change of pH and analyzing the pH measurements for the subject relative to the reference pH or a reference degree of change of pH. The step of evaluating the pressure pattern may comprise the step of determining if the subject's pressure pattern is substantially similar to the reference pressure pattern, wherein the reference pressure pattern is derived from a gastroparetic control group.

In another aspect, the invention provides a method of evaluating gastroparesis comprising the steps of providing an ingestible capsule having a pH sensor, having a subject ingest the capsule, recording pH measurements from the sensor as a function of time as the capsule moves through at least a portion of the gastrointestinal tract of the subject, determining the capsules position at a junction between the stomach and the small bowel of the subject as a function of the pH measurements, determining a transit time of the capsule between the time the capsule is ingested by the subject and the time the capsule is determined to be at the position, providing a reference transit time, and evaluating the transit time relative to the reference transit time.

The method may further comprise the step of having the subject ingest a low fat meal with ingestion of said capsule. The step of evaluating transit time relative to the reference transit time may comprise the step of determining whether the transit time is greater than or less than the reference transit time, and the reference transit time may be about 5 hours.

Accordingly, the general object is to provide a method for evaluating whether a subject has gastroparesis using an ingested capsule.

Another object is to provide a method of diagnosing gastroparesis with an ingested capsule.

Another object is to provide a method of evaluating a subject for gastroparesis based on pressure patterns derived from a capsule passing through the subject's gastrointestinal tract.

Another object is to provide a method for evaluating a subject for gastroparesis based on transit time of a capsule passing through one or more segments of the gastrointestinal tract.

Another object is to provide a method for diagnosing gastroparesis using transit times as determined by a pH sensor and/or a pressure sensor as a capsule passes through the gastrointestinal tract.

Another object is to provide a method of diagnosing gastroparesis in a non-invasive manner suitable for the office setting.

These and other objects and advantages will become apparent from the foregoing and ongoing written specification, the drawings, and the claims.

### BRIEF DESCRIPTION OF THE DRAWINGS

FIG. 1 is a prior art graphical view of pH readings taken by a radio telemetry capsule passing through the gastrointestinal tract. FIG. 1 also shows various segments of the gastrointestinal tract.

FIG. 2 is a graph of pH versus time taken by a capsule passing through the gastrointestinal tract.

FIG. 3 is a graph of pressure over the same period of time shown in FIG. 2 taken by the capsule.

FIG. 4 is a graph of the number of contractions during five minute intervals over the same period of time shown in FIG. 3.

FIG. 5 is a graph of the normalized relative motility index for five minute intervals over the same period of time shown in FIG. 2.

FIG. 6 is a graph of pH, pressure and motility index around passage through the ileo-caecal junction.

FIG. 7 is a sectional view of an ingestible capsule for providing pressure and pH data in FIGS. 2-3.

FIG. 8 is a graph of pressure versus time for a healthy control subject between one hour before and one hour after gastric emptying.

FIG. 9 is a graph of pressure versus time for a gastroparetic subject between one hour before and one hour after gastric emptying.

FIG. 10 is a bar graph comparing healthy subjects with gastroparetic subjects using the average total area under the curve of pressure measurements for three twenty minute intervals starting sixty minutes prior to gastric emptying.

FIG. 11 is a bar graph comparing healthy subjects with gastroparetic subjects using the average number of contractions for three twenty minute intervals starting sixty minutes prior to gastric emptying.

FIG. 12 is a graph showing the relationship between scintigraphic emptying of a meal and gastric emptying time of a capsule. The initial upper tracing and axis on the left show the percentage of the meal remaining over time as measured by scintigraphy. The initial lower tracing and axis on the right show the pH as measured by the capsule.

### DESCRIPTION OF PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

A method is provided for evaluating a subject for gastroparesis using an ingestible capsule as a function of pressure readings, pH readings taken by the ingested capsule and/or transit time. A capsule 20 is ingested by a subject and readings from sensors on the capsule are taken as the capsule passes through the gastrointestinal tract of the subject. Data from the pressure sensor and pH sensor are collected and analyzed by comparison to one or more reference templates to evaluate the subject for gastroparesis.

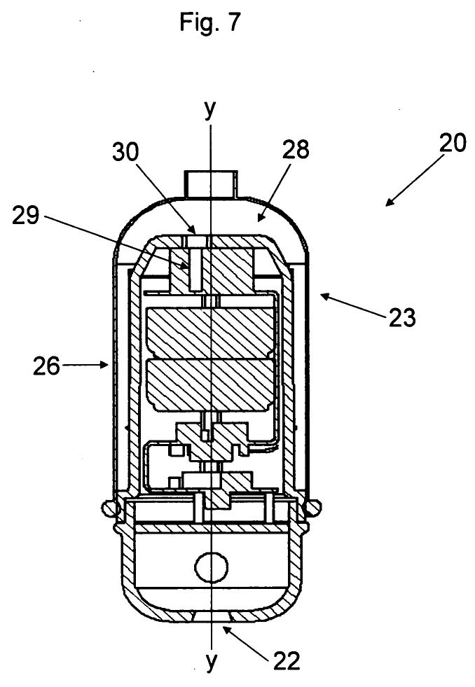

As shown in FIG. 7, capsule 20 is an elongated ellipsoid-shaped device, somewhat resembling a medicament capsule. The capsule generally has a hard shell or casing which houses the transmitting electronics, battery compartment and sensors. Capsule 20 is adapted to be ingested or otherwise positioned within a tract to sense both pressure and pH within the tract and to transmit such readings. As shown, capsule 20 is generally a cylindrical member elongated about axis y-y and having generally rounded closed ends. The capsule is generally provided with an outer surface to facilitate easy swallowing of the capsule.

Capsule 20 includes a pressure sensor assembly 23 comprising a flexible sleeve 26 affixed to the shell of the capsule and defining a chamber 28 between the shell and the sleeve. A pressure sensor 29 is operatively arranged to sense pressure within chamber 28 and communicates with the chamber through a fluid port 30 at one end of the shell of the capsule. As shown, the pressure sleeve 26 of capsule 20 extends from a point below the middle of the capsule up over the top end of the capsule. Capsule 20 also includes a temperature sensor.

On the opposite end of capsule 20 to pressure sensor 23 is pH sensor 22. In the preferred embodiment, pH sensor 22 is a conventional ISFET type pH sensor. ISFET stands for ion-selective field effect transistor and the sensor is derived from MOSFET technology (metal oxide screen field effect transistor). A current between a source and a drain is controlled by a gate voltage. The gate is composed of a special chemical layer which is sensitive to free hydrogen ions (pH). Versions of this layer have been developed using aluminum oxide, silicon nitride and titanium oxide. Free hydrogen ions influence the voltage between the gate and the source. The effect on the drain current is based solely on electrostatic effects, so the hydrogen ions do not need to migrate through the pH sensitive layer. This allows equilibrium, and thus pH measurement, to be achieved in a matter of seconds. The sensor is an entirely solid state sensor, unlike glass bulb sensors which require a bulb filled with buffer solution. Only the gate surface is exposed to the sample.

In the preferred embodiment, the capsule transmits sensed data at about 434 MHz and measures 26.8 mm long by 11.7 mm in diameter. A portable data receiver worn by the subject receives and stores data transmitted by the capsule. Software performs data analysis and presents a graphical data display of pH, pressure and temperature readings for analysis. After activation and ingestion, the capsule senses and transmits data for at least 120 hours after activation. The pH, pressure and temperature data are transmitted from within the GI tract to the data receiver. In the preferred embodiment, the range and accuracy of the sensors are generally 0.5 to 9.0 pH units with an accuracy of ±0.5 pH units, 0 to 350 mmHg with an accuracy of 5 mmHg, or 10% above 100 mmHg, and 25° to 49° C. with an accuracy of ±1° C. The data receiver contains rechargeable batteries and when seated in a docking station allows for battery charging and data download. Data is downloaded from the data receiver through the docking station via USB connection to a Windows PC compatible laptop.

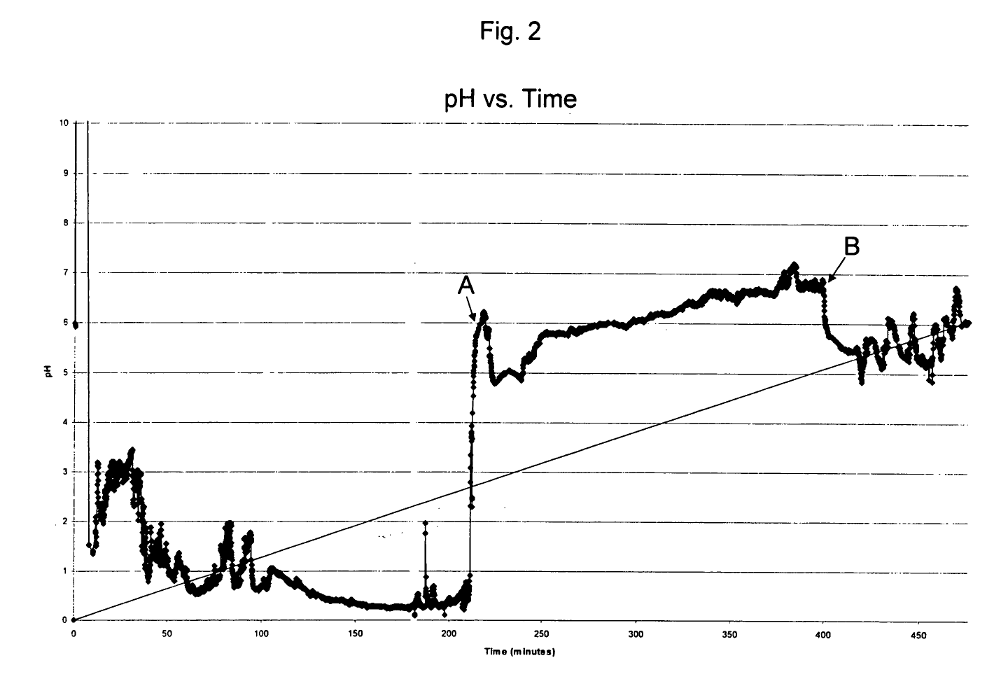

The pH readings from the ingested capsule are plotted against time, as shown in FIG. 2. Based on reference data, a substantial variation or increase in pH, generally indicated at A, indicates passage of the capsule from the acidic antrum to the alkaline duodenum, often referred to as gastric emptying. Thus, based on the pH measurements taken by the capsule, its transition from the stomach to the small bowel can be determined as a function of time. The elapsed time from ingestion to this transition is calculated. In the preferred embodiment, this location is marked as the point at which the pH abruptly rises more than 3 pH units from baseline pH to a pH of greater than 4.

Based on this determination, the capsules gastric emptying or residence time may be determined. Gastric emptying time (GET) of the capsule is the duration of time from the capsule's ingestion to the point at which the foregoing pH rise is determined.

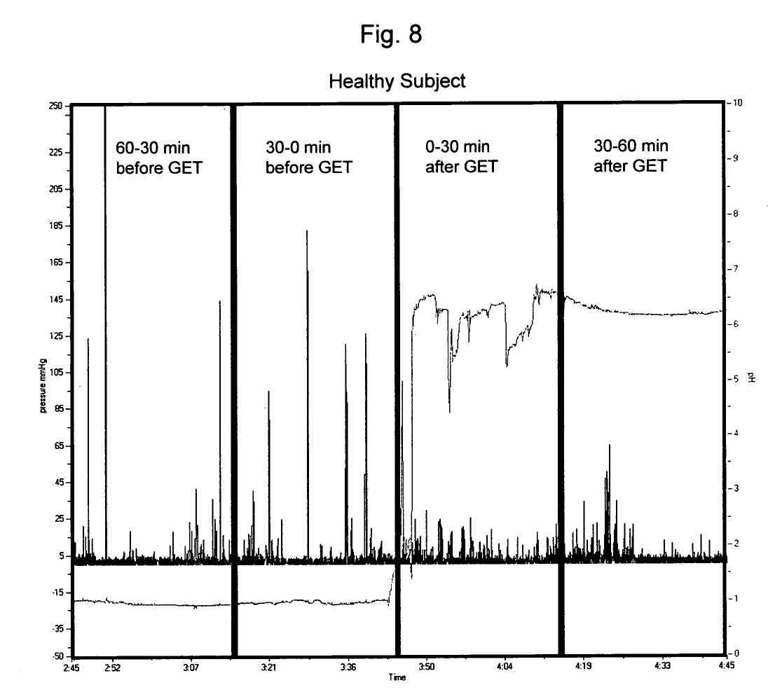

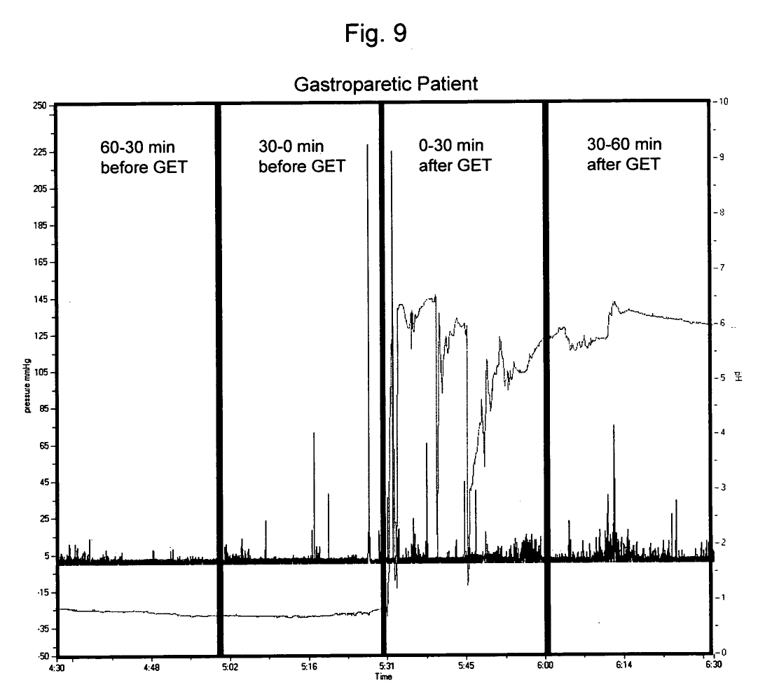

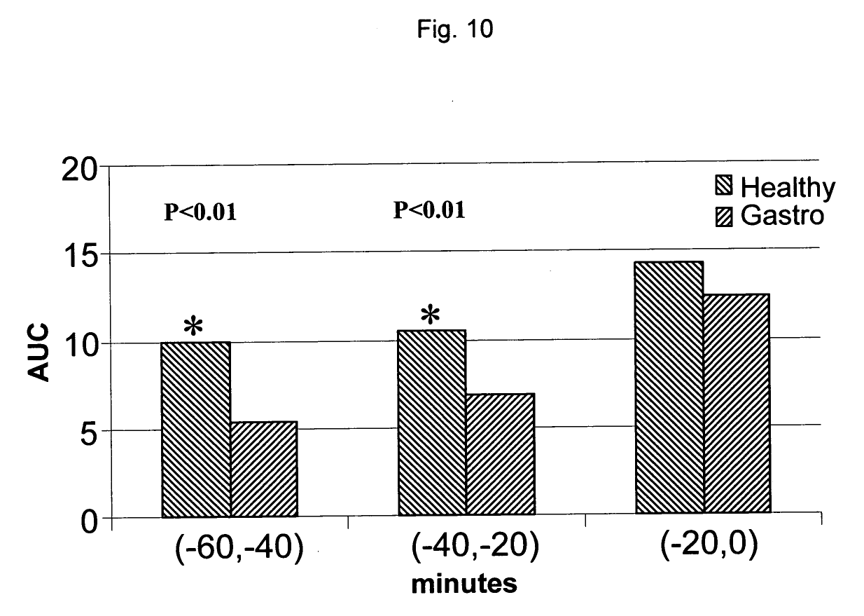

In the preferred embodiment, pressure patterns derived from pressure measurements taken by the capsule during a period of time before and after the capsule has transitioned from the stomach to the small bowel are also used to evaluate the subject for gastroparesis, or delayed gastric emptying. As shown in FIG. 8, with reference to the computer generated time stamp designating gastric emptying of the capsule, pressure data recorded by the capsule for the time period from 1 hour before gastric emptying to one hour after gastric emptying for a healthy non-gastroparetic control group is provided as a reference pressure pattern. As shown in FIG. 9, pressure data for the same time period shown in FIG. 8 is recorded for a subject using the capsule. The pressure data is then analyzed. This pressure data is divided into thirty-minute intervals, as shown in FIGS. 8 and 9. These intervals are 30-60 minutes before gastric emptying, 0-30 minutes before gastric emptying, 0-30 minutes after gastric emptying, and 30-60 minutes after gastric emptying. While FIGS. 8 and 9 show data divided into thirty minute intervals, twenty-minute intervals may be used as an alternative, as further described with reference to FIG. 10. In this case, the intervals would be 40-60 minutes before gastric emptying, 20-40 minutes before gastric emptying, 0-20 minutes before gastric emptying, 0-20 minutes after gastric emptying, 20-40 minutes after gastric emptying and 40-60 minutes after gastric emptying.

In the preferred embodiment, the pressure data from the subject is conditioned to distinguish real contraction data from artifacts or “noise” within the data set, as well as to discount physiologically improbable values. In the preferred embodiment, both concerns are addressed as part of a process which inspects each data value in the pressure measurement data set provided by the capsule. Because the conditioning utilizes constant minimum and maximum threshold values to determine and eliminate data spikes and artifacts, the input pressure data is baseline compensated. As mentioned above, the pressure data is then conditioned by filtering out those sets of data points or contractions whose peaks are above a predetermined threshold or limit. In the preferred embodiment, this threshold is about 200 mmHg. In addition, those contraction patterns whose peaks are less than a predetermined threshold or limit are also filtered out. In the preferred embodiment, this minimum threshold is about 9 mmHg. Thus, in the preferred embodiment the process considers a set of baseline compensated pressure measurements and begins evaluating each value in linear sequence from beginning to end. If a point is found to exceed the defined maximum, then the high value or spike is removed with its associated ascending and descending artifact values by traversing the data set both behind and ahead of the detected spike and zeroing the spike and any associated values, until either its termination or a new contraction is detected. The determination that an artifact has terminated is defined as any data point below a minimum pressure value. Contrarily, finding the next contraction from the high value is based on the detection of three consecutive ascending values, which is interpreted as an ascent in pressure, indicating the edge of a different contraction. Thus, in determining, for example, the area under the curve for a given time interval, a pressure point is included in the calculation only if its value is greater than or equal to the sum of the baseline pressure and the minimum threshold and is below the sum of the baseline pressure and the maximum threshold.

FIG. 10 is a bar graph which shows the average area under the curve for three twenty-minute time intervals before gastric emptying of the capsule. The reference values for the healthy control group are shown on the left. Readings for a gastroparetic subject is shown on the right. As seen in FIG. 10, the time periods 40-60 minutes and 20-40 minutes both show statistically discernible variations between the healthy subject and the gastroparetic subject, with the gastroparetic subject having an area under the curve pressure pattern significantly lower than the reference. In the preferred embodiment, p values that are equal to or less than 0.05 indicate a significant difference.

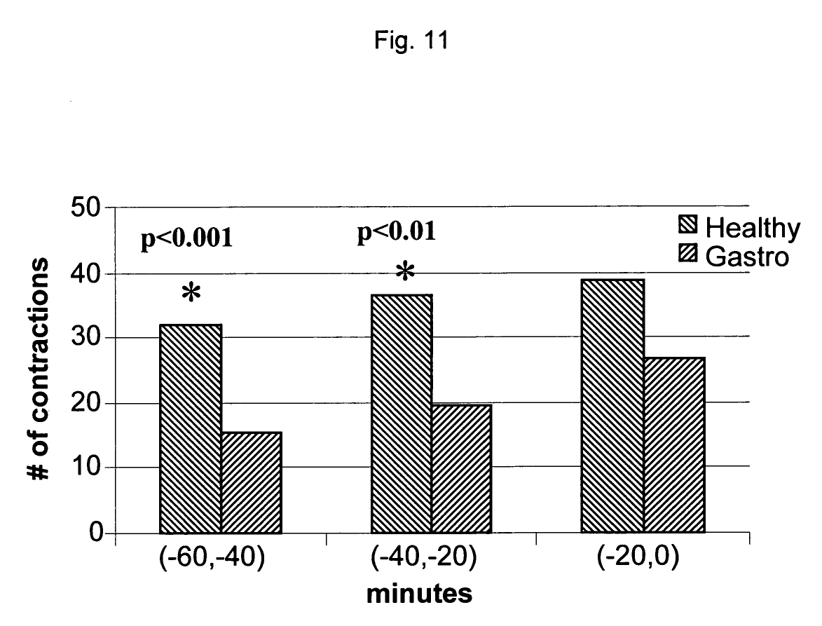

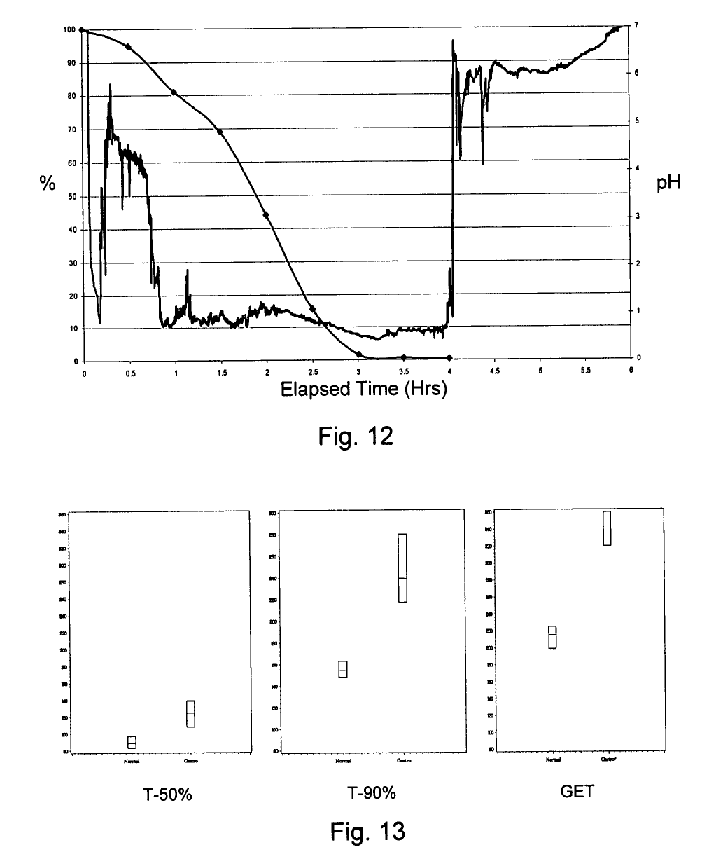

FIG. 11 is a bar graph showing the average number of contractions over a baseline for the same time periods as in FIG. 10. The baseline in the preferred embodiment is about 9 mmHg. Again, the time periods 40-60 minutes before gastric emptying and 20-40 minutes before gastric emptying have statistically discernible variations between healthy and gastroparetic subjects, with the gastroparetic subject having an average number of contractions significantly lower than the referenced healthy control group.

After an overnight fast, 104 subjects (66 healthy and 38 gastroparetic) swallowed the capsule after an Eggbeater meal. Pressure, temperature and pH data were recorded and downloaded into a computer for analysis. The gastric emptying time (GET) was measured as the difference between the time of ingestion to a sudden and sustained rise of pH to greater than 4, and at least 3 pH units above baseline, which was correlated to gastric emptying or the location at the transition between the stomach and small bowel. As described above, the number of gastric contractions and the area under the curve (AUC) of pressure for 20 minute intervals in the last 1 hour of GET were calculated. A two tailed unequal variance t-test was used for statistical analysis, and a p<0.05 was considered significant. Ninety-five percent confidence intervals (CI) were also calculated. Tables 1 and 2 below indicate that, with means and 95% CI, gastroparetics had less contractions and a lower AUC in the last one hour of GET.

TABLE 1
Number of gastric contractions over a baseline recorded by
the capsule for the last one hour of GET in healthy
controls and gastroparetic patients.

| Time | Healthy | Gastroparetics (n = 38) | |
|---|---|---|---|
| (minutes | Controls | Idiopathic (n = 22) | |
| before GET) | (n = 66) | Diabetic (n = 16) | P value |
| 60-40 | 31.8 | 15.3 (8.3-22.3) | 0.00047 |
| | (26.5-37.1) | 17.7 (6.4-29.0) | 0.03485 |
| | | 12.0 (5.7-18.3) | 0.00003 |
| 40-20 | 36.6 | 19.7 (11.0-28.4) | 0.00363 |
| | (29.8-43.0) | 17.5 (10.6-24.4) | 0.00028 |
| | | 22.6 (4.0-42.2) | 0.18792 |
| 20-0 | 39.0 | 26.6 (16.3-36.9) | 0.05626 |
| | (32.4-45.6) | 32.0 (16.4-47.6) | 0.44172 |
| | | 19.2 (7.9-30.6) | 0.00715 |
| 60-0 | 107.1 | 61.6 (39.9-83.3) | 0.00133 |
| | (91.7-132.5) | 67.3 (47.0-97.6) | 0.00283 |
| | | 53.9 (22.6-85.2) | 0.00673 |

TABLE 2
Area under the curve for pressure recorded
by the capsule for the last one hour of GET in healthy
controls and gastroparetic patients.

| Time | Healthy | Gastroparetics (n = 38) | |
|---|---|---|---|
| (minutes | Controls | Idiopathic (n = 22) | |
| before GET) | (n = 66) | Diabetic (n = 16) | P value |
| 60-40 | 9.9 | 5.4 (3.2-7.6) | 0.00275 |
| | (8.1-11.7) | 6.2 (2.7-9.7) | 0.071707 |
| | | 4.3 (2.0-6.6) | 0.000633 |
| 40-20 | 10.5 | 6.8 (4.7-8.9) | 0.006616 |
| | (9-12) | 7.2 (4.2-10.2) | 0.060985 |
| | | 6.2 (3.2-9.2) | 0.020153 |
| 20-0 | 14.3 | 12.3 (8.7-15.8) | 0.349523 |
| | (12.4-16.2) | 14.5 (10-19) | 0.955135 |
| | | 9.4 (3.4-15.4) | 0.14172 |
| 60-0 | 34.8 | 24.5 (17.8-31.2) | 0.01352 |
| | (30.7-38.9) | 27.9 (18.6-37.2) | 0.194014 |
| | | 19.9 (10.5-29.3) | 0.010134 |

Thus, in the preferred embodiment a subject is evaluated for gastroparesis by comparing data recorded by a capsule as it moves through the gastrointestinal tract of the subject with a reference template or model. While in the preferred embodiment the pressure patterns for the time intervals 40-60 minutes before gastric emptying and 20-40 minutes before gastric emptying are used for purposes of comparison with the reference pattern, it is contemplated that other time periods may be used. For example, gastric contractions for the subject and the reference may be compared during the time interval of 30-60 minutes before gastric emptying. Alternatively, the window may be as large as the entire gastric residence time. Gastroparetics also have a statistically significant lower number of gastric contractions over a baseline in this time period when compared to a healthy control group, with p values of less than 0.02. In a study of 21 healthy subjects and 16 gastroparetic subjects, the healthy subjects had an average number of contraction per minute, or frequency of contractions, of about 1.23 during this period, while gastroparetic subjects had an average number of contraction above a 9 mmHg baseline per minute of about 0.74 during the same time period, with a p value of about 0.013.

Also, while a decrease in pressure patterns (such as frequency of contractions or motility index) compared to a template derived from pressure readings from non-gastroparetic controls is used in the preferred embodiment to evaluate a subject for gastroparesis, it is contemplated that alternatively the template may be derived from a gastroparetic control group, and similarities, rather than differences, between pressure patterns of the subject and the reference template may be used to evaluate the subject for gastroparesis. Furthermore, as indicated in Tables 1 and 2, these pressure patterns may be used to distinguish between gastroparetic subgroups, such as idiopathic and diabetic.

In the preferred embodiment, transit time in the stomach is used to supplement the information available for evaluating a subject for gastroparesis. In an alternative embodiment, transit time of the capsule in the stomach may be used alone to evaluate gastroparesis. In this embodiment, the non-digestible capsule 20 is used to determine GET and evaluate a subject for gastroparesis. GET is determined with the capsule based on the duration of time from the capsules ingestion to the point at which the capsule indicates an abrupt pH increase greater than 3 pH units from a baseline pH to a pH greater than 4. In the preferred embodiment, if this time is determined to be greater than about 300 minutes after ingestion of a standardized 255 k cal low fat meal and the capsule, then the subject is evaluated as having a gastroparetic condition.

A study was conducted to assess the correlation between an ingestible capsule's GET and the gastric emptying scintigraphy (GES) technique presently used to measure gastric emptying time, and to determine whether the capsule could discriminate healthy subjects from gastroparetics. Eighty-six healthy subjects and 60 gastroparetics were studied simultaneously with the capsule and GES. After overnight fast, subjects swallowed the capsule and ingested a ^{99m}Tc-SC radio-labeled low fat (255 k cal) meal. Images were obtained at 30 minute intervals for 6 hours. GET was determined for each subject. Correlations between capsule GET and GES time to 50% emptying (T-50%) and time to 90% emptying (T-90%) were performed. Correlation between capsule GET and GES T-90% was 0.82±0.06, and correlation for capsule GET and GES T-50% was 0.66±0.15. The diagnostic accuracy, as assessed by ROC, between gastroparetics and healthy subjects was 0.83 for capsule GET and 0.85 for T-90% (not statistically different) and 0.77 for T-50%. The cutoff time for capsule GET that maximizes both sensitivity and specificity for diagnosis of gastroparesis was 300 min, giving 86% sensitivity and 92% specificity. Thus, this new capsule based method correlates with T-90% GES emptying and discriminates between healthy and gastroparetic subjects, offering an efficient, ambulatory alternative to scintigraphy.

A ROC curve was used to examine the diagnostic utility the two tests in discriminating healthy normals and patients with gastroparesis. The area under the curve (AUC), and its corresponding 95% bootstrap confidence interval, was used as the primary measure of diagnostic utility. The optimal sensitivity versus specificity cutoff for diagnosing gastroparesis versus normal was taken to be the upper leftmost point on the ROC curve. The gold standard scintigraphy diagnosis of gastroparesis was based on previous history of disease. An additional analysis was performed using a refined gold standard definition of disease, which combined history of the disease with scintigraphy confirmation from the day of the test.

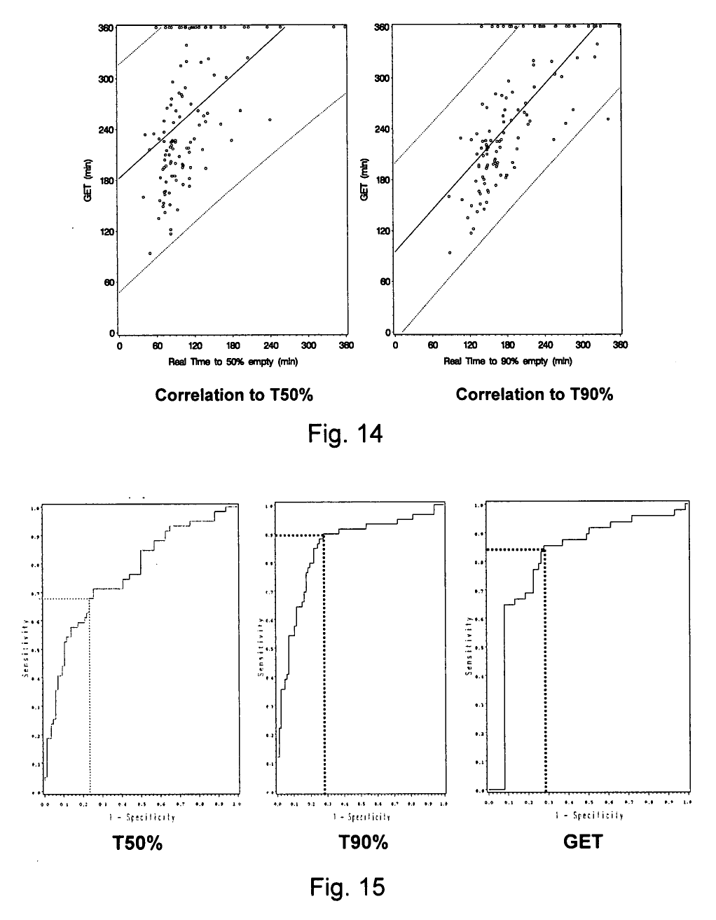

FIG. 12 shows an example of the relationship between gastric emptying of the scintigraphic meal simultaneously with the pH tracing for the capsule. This demonstrates the emptying of the indigestible capsule when the capsule leaves the acid antrum into the alkaline duodenum. The radio-labeled meal empties completely before the capsule empties.

The median and 95% confidence interval times for T-50% emptying and T-90% emptying measure by scintigraphy and the GET measured by the capsule in the 125 healthy subjects and patients with gastroparesis are shown in Table 3 below.

TABLE 3
Median Emptying (minutes) with Corresponding
95% Confidence Intervals

| | Median T50% | Median T90% | Median Capsule |
|---|---|---|---|
| Healthy | 89 (83, 97) | 154 (148, 163) | 215 (199, 225) |
| Gastros | 124 (108, 139) | 239 (217, 280) | 360 (320, >360) |

In the healthy subjects, the median T-50% was 89 min, T-90% was 154 minutes, and capsule GET was 215 minutes. In patients with gastroparesis, the median T-50% was 123 minutes, T-90% was 239 minutes, and capsule GET was 360 minutes. Measurements times of T-50%, T-90% and capsule GET from the healthy subjects compared the gastroparetic subjects were statistically different from each other (p<0.05).

Table 4 below summarizes the sensitivity and specificity of T-50% GES, T-90% GES, and capsule GET based on the optimal cut-points from the ROC curve with the corresponding AUC values (c statistic).

TABLE 4
Sensitivity and Specificity Values Based on the
Optimal Cutoff for History of Gastroparesis

| Parameter | GET Corr. | Sensitivity | Specificity | AUC (905% CI) |
|---|---|---|---|---|
| T-50% | 0.50 | 0.69 | 0.75 | 0.77 (0.74, 0.87) |
| T-90% | 0.82 | 0.90 | 0.72 | 0.85 (0.80, 0.90) |
| GET | | 0.85 | 0.72 | 0.83 (0.74, 0.87) |

In the preferred embodiment, the optimal cutoff point for capsule GET to discriminate between healthy subjects and gastroparetic patients is 300 minutes as determined from the sensitivity and specificity of this analysis.

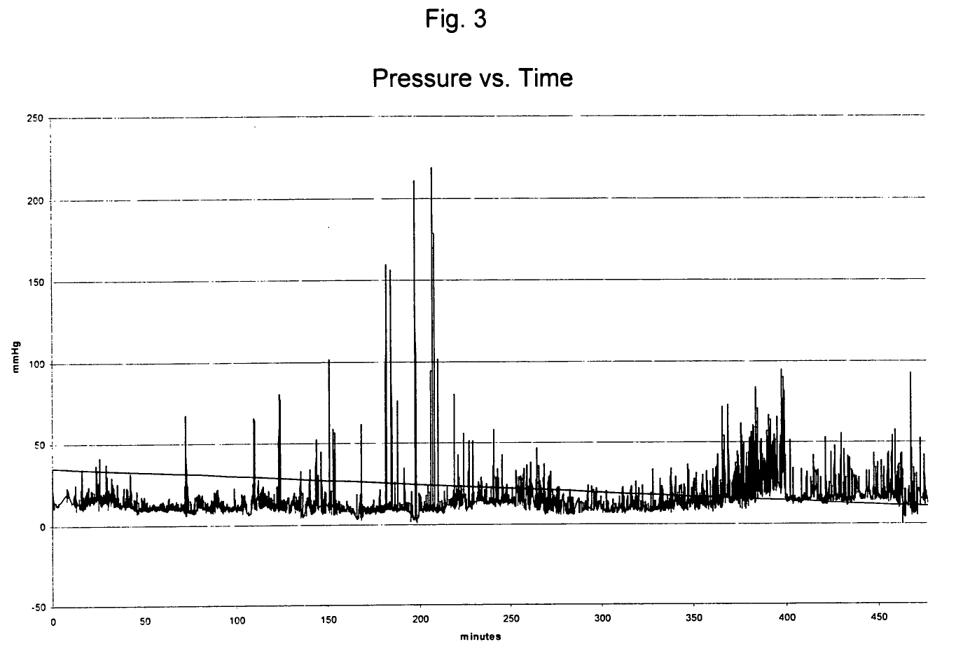

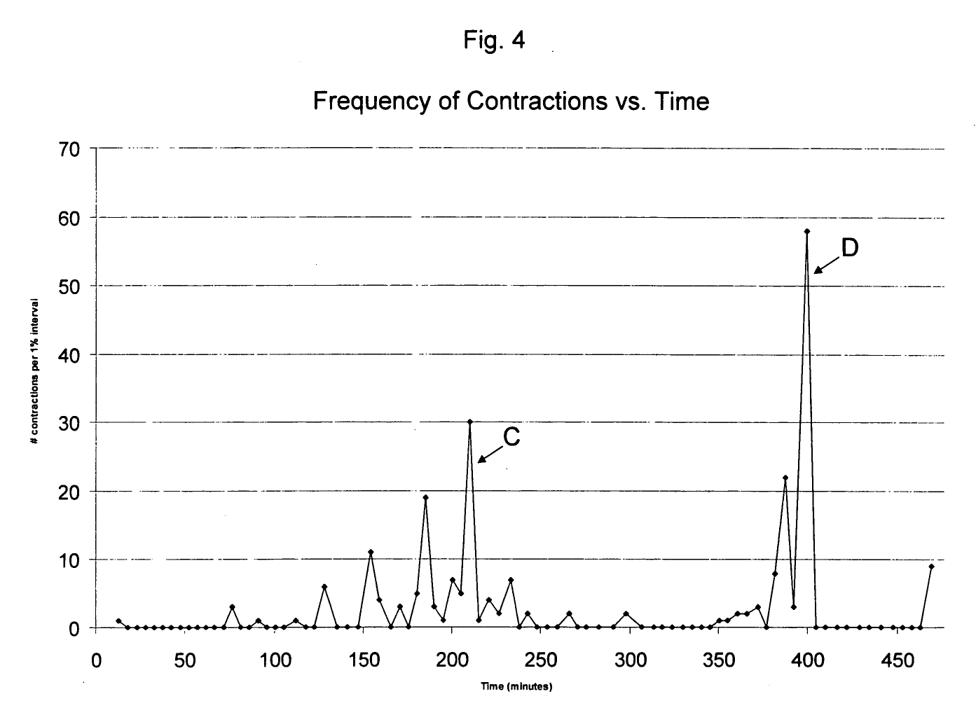

Transit time in the small bowel may also be used to supplement the information available for evaluating a subject for gastroparesis. A latter variation in pH, indicated at B in FIG. 2, suggests movement of the capsule from the ileum to the caecum. It has been found that this significant pH drop is seen hours after gastric emptying and is due to the capsule moving from the ileum to the caecum, a transition referred to as the ileo-caecal junction. In this embodiment, not only is a variation in pH patterns used to determine that the capsule is at the junction between the stomach and small bowel or at the ileo-caecal junction, but an associated change in pressure pattern is also employed. Average pressure readings from the capsule plotted against transit time are shown in FIG. 3. The number of contractions over a given time interval, five minutes in the preferred embodiment, plotted against the same overall time period are shown in FIG. 4. In the preferred embodiment, a contraction is designated by an increase in pressure over 10 mmHg and the subsequent return below 10 mmHg. However, it is contemplated that gastrointestinal contractions may be determined based on other variations in pressure or baselines other than 10 mmHg.

As shown in FIG. 4, a variation in the frequency of contractions was generally found to occur, as indicated at C, at a time corresponding to the gastric emptying suggested by the graph of pH shown in FIG. 2. This correlation between the variation in frequency of contractions C and the variation in pH A is used as a reference to confirm that the capsule has moved from the stomach to the small bowel. A further and more substantial variation in frequency of contractions occurs, as indicated at D, at a time corresponding to the ileo-caecal junction suggested by the graph of pH shown in FIG. 2. This correlation between the variation in frequency of contractions D and the variation in pH B is used as a reference to determine that the capsule has moved from the ileum to the caecum of the subject.

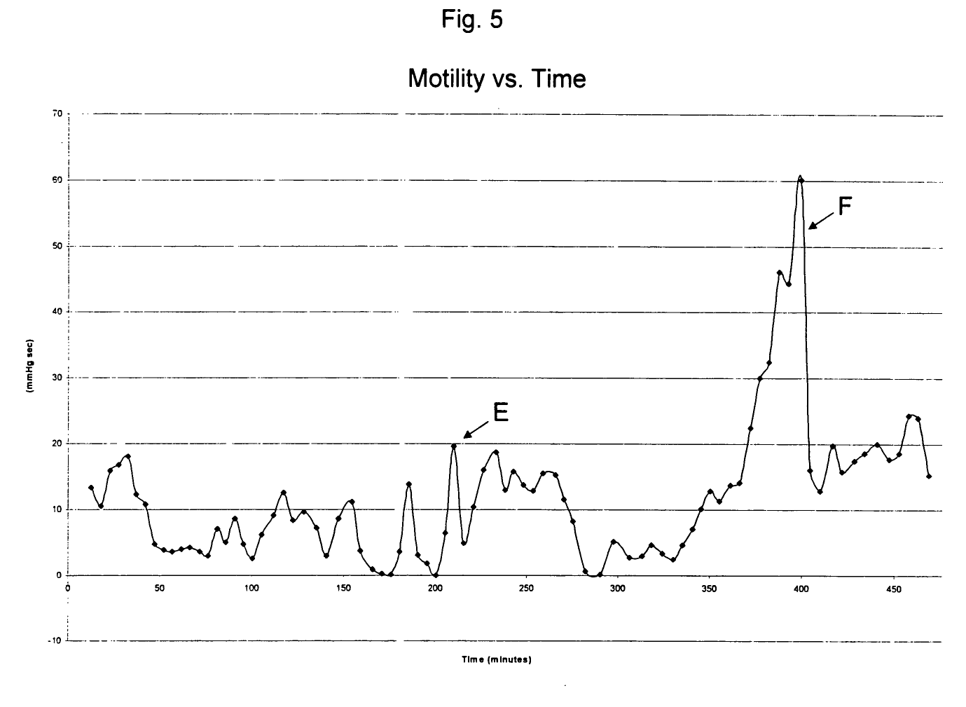

FIG. 5 is a plot of the normalized relative motility index at five minute intervals versus time. Each data point is the area under the curve of the graph of pressure shown in FIG. 3 for five minute intervals. Motility index as used herein is the area under the curve (or the integral of pressure over a time region) divided by the size of the time region. While a five minute time region is used in this graph, other time periods may be employed. Plotted against transit time, generally a substantial variation occurs, indicated at F, at substantially the same time as the variation B in pH. This variation in motility index is used in the preferred embodiment as a reference to confirm that the capsule has moved from the ileum to the caecum of the subject. Also, a variation in motility index indicated at E may be used as a reference with pH variation A to confirm that the capsule has moved from the stomach to the small intestine.

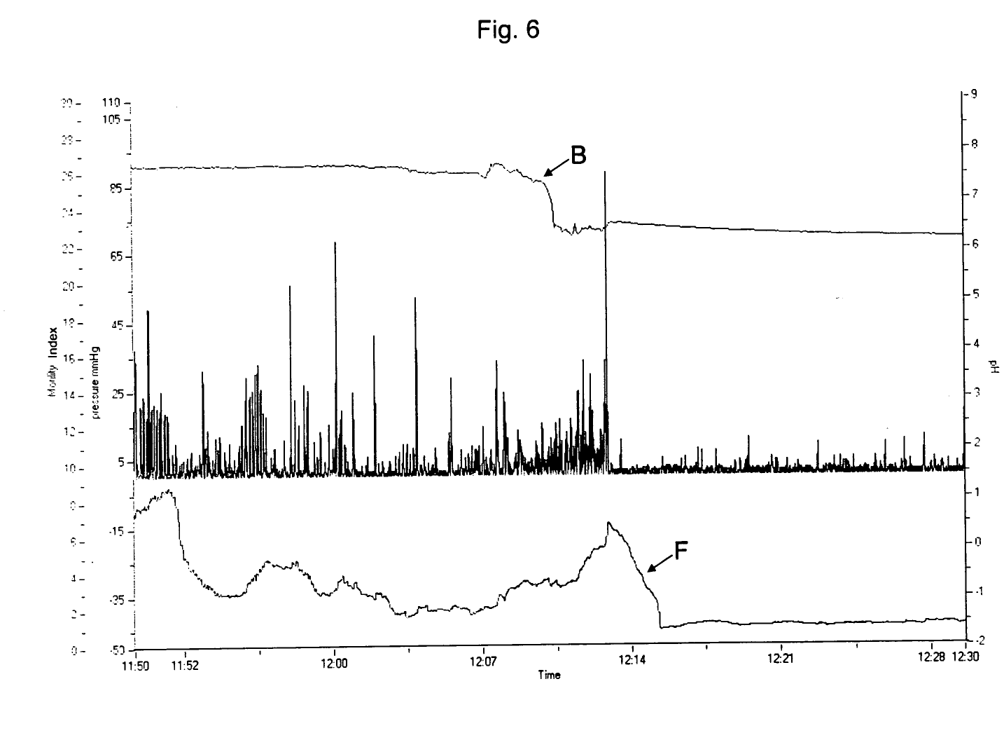

FIG. 6 is a representative graph of raw pH and pressure readings for a subject, together with motility index, for the twenty minutes prior to passing through the ileo-caecal junction and twenty minutes after passing through the ileo-caecal junction. As shown, the motility index stabilizes and flattens out after passage through the ileo-caecal junction.

By basing location on both pH and pressure patterns, one can more accurately determine the movement of ingested capsule 20 from one segment of the gastrointestinal tract to a second segment of the gastrointestinal tract of a subject. In comparing patterns from a subject with the reference templates for both pH and pressure, if there is a correlation between a variation in pH B and a variation in frequency of contractions D and/or motility F, then a determination of the capsule's location may be more accurate.

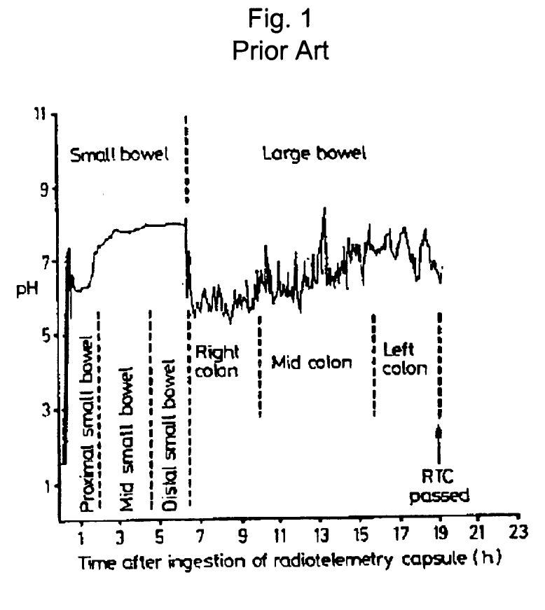

The patterns indicate that the intraluminal environment of the gastrointestinal tract as it transitions from the small intestine into the colon changes. The caecum, as compared to the distal ileum, is a less contractile reservoir where colonic bacteria cause an acidic change in pH. Thus, in the preferred embodiment, capsule 20 is ingested by the subject and pH readings and pressure readings are taken and compared as indicated above. Certain pH reference values are known in the prior art, as shown in FIG. 1. In addition, reference patterns, from which reference templates FIG. 2-5 were derived, were formed from capsule testing data. One hundred four volunteers swallowed an ingestible capsule having a pH sensor and a pressure sensor after an overnight fast, together with a standardized meal and 100 cc's of water. As indicated above, it was found that a rapid pH change from acidic to alkaline (greater than 4 and at least a 3 unit rise from baseline gastric pH) marked the emptying of the ingested capsule from the stomach into the duodenum or small bowel. On the capsule's recordings, approximately 5.5 hours after the capsule's gastric emptying, a drop in pH of greater than 1 unit for more than 5 minutes was generally found. The frequency and the amplitude of contractions were analyzed from 30 minutes before the beginning of the pH drop to 30 minutes after. These parameters were then compared by two-sample unequal variance t test. The results of the test showed that average time from the gastric emptying to the pH drop was 5 hours and 23 minutes. The frequency of contractions for the 30 minutes before the pH drop was shown to be 3.9 contractions per minute (95% CI 3.99±0.014), and for the 30 minutes after the drop was 2.1 contractions per minute (95% CI 2.1±0.01), p<0.0001. The mean amplitude of contractions was no different between the time periods chosen (19.6 mmHg before, 19.4 mmHg after the pH drop, p=0.8). The motility index for the 30 minutes before the pH change was 1.54 and the motility index for the 30 minutes after the pH change was 0.91, p<0.0001.

Thus, readings from a subject may also be compared to reference templates to determine the location of the capsule, with a change in pH and a change in either frequency of contractions or motility index correlating with the variations in the template used to determine that location. By using patterns based on both pH and pressure, location is more accurate because changes in pH based on bacterial overgrowth or malignancies in the gastrointestinal tract are not assumed to be a transition from one segment to a second segment if they are not accompanied by a corresponding variation in either the frequency of contractions or motility index.

With the determination that the capsule has passed from the stomach to the small bowel and then through the ileo-caecal junction, transit time through the small bowel is ascertained. Transit time through the colon can then be determined as well, as the time from passage through the ileo-caecal junction to discharge of the capsule. These times may then be used as additional information in the evaluation of the subject for gastroparesis.

The present invention contemplates that many changes and modifications may be made. Therefore, while the presently-preferred form of the improved method has been shown and described, and a number of alternatives discussed, persons skilled in this art will readily appreciate that various additional changes and modifications may be made without departing from the spirit of the invention, as defined and differentiated by the following claims.

What is claimed is:

1. A method of evaluating gastroparesis comprising the steps of:
providing an ingestible capsule having a pH sensor and a pressure sensor;
having a subject ingest said capsule;
recording pH measurements from said pH sensor as a function of time as said capsule moves through at least a portion of the gastrointestinal tract of said subject;
recording pressure measurements from said pressure sensor as a function of time as said capsule moves through at least a portion of said gastrointestinal tract of said subject;
determining said capsules location at a position in said gastrointestinal tract;
deriving a pressure pattern as a function of time and said pressure measurements;
providing a reference pressure pattern; and
analyzing said subject's pressure pattern relative to said reference pressure pattern to evaluate said subject with respect to gastroparesis.

2. The method set forth in claim 1, wherein said position is the junction between the stomach and the small bowel of said subject.

3. The method set forth in claim 1, wherein said pressure pattern is the number of contractions relative to a baseline for a given time interval.

4. The method set forth in claim 1, wherein said pressure pattern is the area under the curve of said pressure measurements for a given time interval.

5. The method set forth in claim 1, wherein said pressure pattern is amplitude.

6. The method set forth in claim 1, wherein said step of evaluating said subject comprises diagnosing gastroparesis.

7. The method set forth in claim 3, wherein said step of analyzing said pressure pattern comprises the step of determining if said subject's pressure pattern is significantly lower than said reference pressure pattern, wherein said reference pressure pattern is derived from a healthy control group.

8. The method set forth in claim 7, wherein said subject's pressure pattern is at least ten percent less than said reference pressure pattern.

9. The method set forth in claim 1, wherein said time is the time said capsule resides in the stomach of said subject.

10. The method set forth in claim 1, wherein said time extends from about one hour before said capsules is determined to be at said position to about the time said capsule is determined to be at said position.

11. The method set forth in claim 1, wherein said step of analyzing said subject's pressure pattern relative to said reference pressure pattern to evaluate said subject with respect to gastroparesis comprises the step of comparing said subject's pressure pattern to said reference pressure pattern for three consecutive twenty minute intervals before and after said capsule is determined to be at said position.

12. The method set forth in claim 1, and further comprising the steps of determining the transit time between a first location and a second location, and evaluating said transit time relative to a reference transit time.

13. The method set forth in claim 12, wherein said first location is the point at which said capsule is ingestible by said subject and said second location is the junction between the stomach and the small bowel of said subject.

14. The method set forth in claim 13, wherein said step of evaluating said transit time relative to a reference transit time comprises the step of determining whether said transit time is greater than or less than said reference transit time.

15. The method set forth in claim 14, wherein said reference said transit time is about 5 hours.

16. The method set forth in claim 1, wherein said step of deriving a pressure pattern as a function of time and said pressure measurements comprises the step of conditioning said recorded pressure measurements.

17. The method set forth in claim 16, wherein said conditioning comprises the step of normalizing said data points by applying a baseline compensation.

18. The method set forth in claim 17, wherein said baseline is about 3 mmHg.

19. The method set forth in claim 16, wherein said conditioning comprises the steps of filtering out data points in said pressure measurements above an upper limit and filtering out data points in said pressure measurements below a lower limit.

20. The method set forth in claim 17, wherein said upper limit is about 200 mmHg and said lower limit is about 9 mmHg.

21. The method set forth in claim 1, and further comprising the steps of deriving a pH pattern as a function of time and said pH measurements and analyzing said pH pattern for said subject and said pressure pattern for said subject relative to a pH reference pattern and a pressure reference pattern to determine said capsule's location at a second position.

22. The method set forth in claim 21, and further comprising the steps of determining transit time between said first position and said second position, and evaluating said transit time relative to a reference transit time.

23. The method set forth in claim 22, wherein said first position is a junction between the stomach and the small bowel of said gastrointestinal tract of said subject and said second position is a junction between the ileum and the caecum of said gastrointestinal tract of said subject.

24. The method set forth in claim 22, wherein said first position is the junction between the ileum and the caecum of said gastrointestinal tract of said subject and said second position is the point at which said capsule is discharged from said gastrointestinal tract of said subject.

25. The method set forth in claim 1, and further comprising the steps of:
deriving a second pressure pattern different from said first pressure pattern as a function of time and said pressure measurements;
providing a second reference pressure pattern; and
analyzing said second pressure pattern variations for said subject relative to said second reference in determining said capsule's location at said position.

26. The method set forth in claim 25, wherein said first pressure pattern is frequency of contractions relative to a baseline over a given time interval and said second pressure pattern is motility index.

27. The method set forth in claim 21, and further comprising the steps of:
deriving a second pressure pattern different from said first pressure pattern as a function of time and said pressure measurements;
providing a second reference pressure pattern; and
analyzing said second pressure pattern variations for said subject relative to said second reference in determining said capsule's location at said second position.

28. The method set forth in claim 27, wherein said first pressure pattern is frequency of contractions and said second pressure pattern is motility index.

29. The method set forth in claim 1, wherein said step of determining said capsules location at a position in said gastrointestinal tract comprises the steps of:
providing a reference pH;
analyzing said pH measurements for said subject relative to said reference pH to determine said capsule's location at said position.

30. The method set forth in claim 1, wherein said step of analyzing said pressure pattern comprises the step of determining if said subject's pressure pattern is substantially similar to said reference pressure pattern, wherein said reference pressure pattern is derived from a gastroparetic control group.

31. A method of evaluating gastroparesis comprising the steps of:
providing an ingestible capsule having a pH sensor;
having a subject ingest said capsule;
recording pH measurements from said pH sensor as a function of time as said capsule moves through at least a portion of the gastrointestinal tract of said subject;
determining said capsules position at a junction between the stomach and the small bowel of said subject as a function of said pH measurements;
determining a transit time of said capsule between the time said capsule is ingested by said subject and the time said capsule is determined to be at said position;
providing a reference transit time; and
evaluating said transit time relative to said reference transit time.

32. The method set forth in claim 31, and further comprising having said subject ingest a low fat meal with said ingestion of said capsule.

33. The method set forth in claim 31, wherein said step of evaluating said transit time relative to said reference transit time comprises the step of determining whether said transit time is greater than or less than said reference transit time.

34. The method set forth in claim 31, wherein said reference transit time is about 5 hours.
